(19) United States
(12) Patent Application Publication
(10) Pub. No.: US 2025/0090628 A1
(43) Pub. Date: Mar. 20, 2025

(54) METHODS AND COMPOSITIONS FOR TREATING HERPES SIMPLEX VIRUS

(71) Applicant: President and Fellows of Harvard College, Cambridge, MA (US)

(72) Inventors: Smita Gopinath, Cambridge, MA (US); Amanda Nd Adams, Cambridge, MA (US)

(21) Appl. No.: 18/890,271

(22) Filed: Sep. 19, 2024

Related U.S. Application Data
(60) Provisional application No. 63/539,200, filed on Sep. 19, 2023.

(51) Int. Cl. A61K 38/16; A61K 31/522; A61K 31/739; A61K 35/747

(52) U.S. Cl. CPC A61K 38/164; A61K 31/522; A61K 31/739; A61K 35/747

(57) ABSTRACT

Provided herein are methods of treating or preventing the transmission of Herpes Simplex Virus (HSV) in a subject, the method comprising administering to the subject a composition comprising a L. crispatus bacteria and/or a peptidoglycan disclosed herein.

### RELATED APPLICATIONS

This application claims a right of priority to and the benefit of U.S. Provisional Application No. 63/539,200, filed on Sep. 19, 2023, which is hereby incorporated by reference herein in its entirety.

### BACKGROUND

Approximately 1 in 1000 newborns in the United States experience a neonatal herpes simplex virus (HSV) infection, resulting from HSV exposure during vaginal delivery. Women with recurrent genital herpes have a risk of vertically transmitting HSV to their neonate. However, women who acquire a genital HSV infection during pregnancy have a higher risk. Additionally, other pathological states that result from HSV infection can affect all populations. For example, herpes encephalitis is the leading cause of lethal encephalitis in the United States and ocular HSV infection is a common cause of blindness in the United States.

Thus, there remains a need for novel therapies for treating HSV as well as preventing the transmission of HSV.

### SUMMARY

The vaginal microbiome is an important determinant of host health and the first barrier encountered by sexually transmitted pathogens during infection. Using a collection of human vaginal microbial strains. Applicant shows that the prominent vaginal strain, Lactobacillus crispatus, reduces infectivity of sexually transmitted pathogen Herpes Simplex Virus (HSV). Reduction of HSV infectivity is species specific, with L. crispatus reducing infection and disease better than gut-associated L. reuteri. Further, active cell metabolism is not required as UV-killed L. crispatus retain the ability to reduce herpes infection. Since one of the most abundant structures on the outside of the L. crispatus cell is peptidoglycan, Applicant assessed whether peptidoglycan could reduce HSV infection.

Applicant found that purified peptidoglycan from bacterial sources reduced herpes infection in vitro and in vivo in a mouse model of genital herpes infection. Mice were susceptible to reinfection, indicating that immunological memory is not activated. Cleavage of the glycosidic linkages in the peptidoglycan chain with lysozyme restored virus infectivity in vitro and in vivo, showing that antiviral effects are dependent on both intact and longer peptidoglycan chains. Such results provide an understanding of the ways that the vaginal microbiome serves as a physical barrier to infection and why some vaginal communities promote better antiviral protection than others.

In some aspects, provided herein are methods of treating or preventing the transmission of Herpes Simplex Viruses (HSV) in a subject, the method comprising administering to the subject a composition comprising a peptidoglycan (e.g., an isolated or purified peptidoglycan). The peptidoglycan may be derived from a bacteria. The bacteria may be a lactobacilli, such as a vaginal lactobacilli or intestinal lactobacilli. In some embodiments, the lactobacilli are Lactobacillus crispatus. In some embodiments, the lactobacilli are Lactobacillus reuteri. In some embodiments, the lactobacilli are Lactobacillus iners. In some embodiments, the lactobacilli are any species or strain listed in Table 1. In some embodiments, the bacteria are any species or strain listed in Table 2.

In some aspects, provided herein are methods of reducing the infectivity of Herpes Simplex Viruses (HSV) in a subject, the method comprising administering to the subject a composition comprising a peptidoglycan (e.g., an isolated or purified peptidoglycan).

In other aspects, provided herein are methods of preventing or treating a Herpes Simplex Viruses (HSV) infection in a subject in need thereof, the method comprising administering to the subject a composition comprising a peptidoglycan (e.g., an isolated or purified peptidoglycan).

The peptidoglycan may be derived from a bacteria. The bacteria may be a lactobacilli, such as a vaginal lactobacilli or intestinal lactobacilli. In some embodiments, the lactobacilli are Lactobacillus crispatus. In some embodiments, the lactobacilli are Lactobacillus reuteri. In some embodiments, the lactobacilli are Lactobacillus iners. In some embodiments, the lactobacilli are any species or strain listed in Table 1.

The basic structure of peptidoglycan contains a carbohydrate backbone of alternating units of N-acetylglucosamine (GlcNAc) and N-acetylmuramic acid, with the N-acetylmuramic acid residues cross-linked to peptides. In some embodiments, a peptidoglycan is at least 10 disaccharide units long. In some embodiments, the peptidoglycan is at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 disaccharide units long.

In some embodiments, the peptidoglycan is no more than 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 disaccharide units long.

In some aspects, provided herein are methods of treating or preventing the transmission of Herpes Simplex Viruses (HSV) in a subject, the method comprising administering to the subject a composition comprising a Lactobacillus bacterium (such as a bacterium from a Lactobacillus crispatus strain listed in Table 1).

In some aspects, provided herein are methods of reducing the infectivity of Herpes Simplex Viruses (HSV) in a subject, the method comprising administering to the subject a composition comprising a Lactobacillus bacterium (such as a bacterium from a Lactobacillus crispatus strain listed in Table 1).

In some aspects, provided herein are methods of preventing or treating Herpes Simplex Viruses (HSV) infection in a subject, the method comprising administering to the subject a composition comprising a Lactobacillus bacterium (such as a bacterium from a Lactobacillus crispatus strain listed in Table 1).

The bacterium may be replication competent (capable of replicating). The bacterium may not be replication competent (e.g., it may be killed bacteria). In some embodiments, the bacterium are lactobacilli are any species or strain listed in Table 1.

Also provided herein are methods of treating or preventing the transmission of Herpes Simplex Virus (HSV) in a subject, the method comprising administering to the subject a composition comprising a lipoteichoic acid (LTA) (e.g., a LTA that is isolated or derived from a L. reuteri or S. aureus bacterium, or any other bacterium disclosed herein, such as a species or strain listed in Tables 1 and 2).

In some embodiments, the HSV is HSV-1 or HSV-2. In some embodiments, the composition is formulated for topical delivery. The composition may comprise a pharmaceutically acceptable carrier. In some embodiments, the method further comprises administering (e.g., conjointly administering) an additional agent for the treatment or prevention of HSV. In some embodiments, the method may comprise administering (e.g., conjointly administering) a Lactobacillus bacterium (e.g., any Lactobacillus bacterium disclosed herein) and a peptidoglycan to a subject.

As disclosed herein, in any embodiment, the Lactobacillus bacterium may be a L. crispatu, L. gasseri, L. jensenii, L. reuteri, L. rhamnosus, or an L. iners bacterium.

Also provided herein are kits comprising (a) a composition comprising a peptidoglycan and (b) an additional agent for the prevention of HSV selected from acyclovir, valacyclovir, or famciclovir. In some embodiments, the peptidoglycan is isolated or derived from L. crispatus bacterium or any other bacterium disclosed herein. The HSV may be HSV-1 or HSV-2.

Additionally, provided herein are kits comprising (a) a composition comprising a lipoteichoic acid and (b) an additional agent for the prevention of HSV selected from acyclovir, valacyclovir, or famciclovir. The lipoteichoic acid may be isolated or derived from L. reuteri bacterium or a S. aureus bacterium, or any other bacteria disclosed herein.

As used herein, an infected subject may be afflicted with one or more pathological manifestations associated with herpes virus infection, such as orolabial herpes, herpetic sycosis (HSV folliculitis), herpes gladiatorum, herpetic whitlow, ocular HSV infection, herpes encephalitis, Kaposi varicelliform eruption (eczema herpeticum), and/or neonatal herpes viral infection.

### BRIEF DESCRIPTION OF THE DRAWINGS

FIG. 1 shows Lactobacilli reduce HSV-2 infection in vitro.

FIG. 2 shows reduction of HSV-2 infection doesn't require active metabolism.

FIG. 3 shows bacterial peptidoglycan reduces HSV-2 infection in vitro.

FIG. 4 shows peptidoglycan reduces HSV-1 infection in vitro.

FIG. 5 shows peptidoglycan reduces HSV-2 infectivity in vivo.

FIG. 6 shows peptidoglycan reduces HSV-2 infectivity in vivo.

FIG. 7 shows that vaginal bacteria may reduce HSV-2 infections.

FIG. 8 shows that dead lactobacilli reduce HSV-2 infections.

FIG. 9 shows addition of purified peptidoglycan reduces HSV-2 infection.

FIG. 10 shows isolated peptidoglycan inhibits HSV-1 infection in fibroblasts.

FIGS. 11 and 12 show peptidoglycan inhibits HSV-1 infection in fibroblasts.

FIG. 13 shows that addition of purified peptidoglycan with HSV-2 increases survival.

FIG. 14 shows peptidoglycan structure.

FIGS. 15-17 show lysozyme treatment partially restores HSV-2 infectivity.

FIGS. 18 and 19 shows lysozyme treatment reduces protectiveness of peptidoglycan.

FIG. 20 shows mice treated with L. crispatus supernatants show improvement in HSV-2 symptoms.

FIG. 21 shows L. reuteri lipoteichoic acid suppresses HSV-2 infection in mice.

FIG. 22 shows S. aureus lipoteichoic acid suppresses HSV-2 infection in vitro.

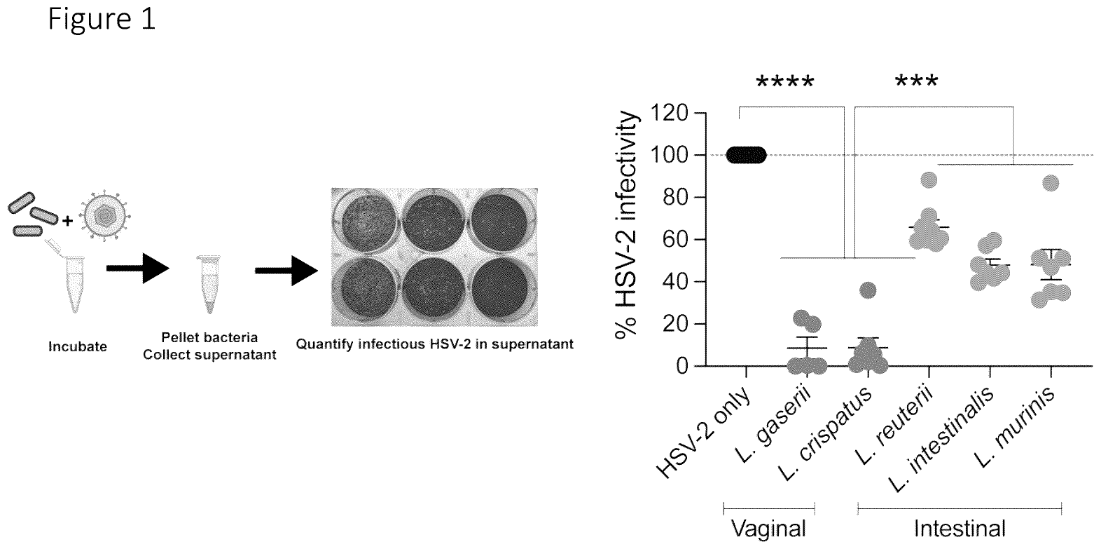

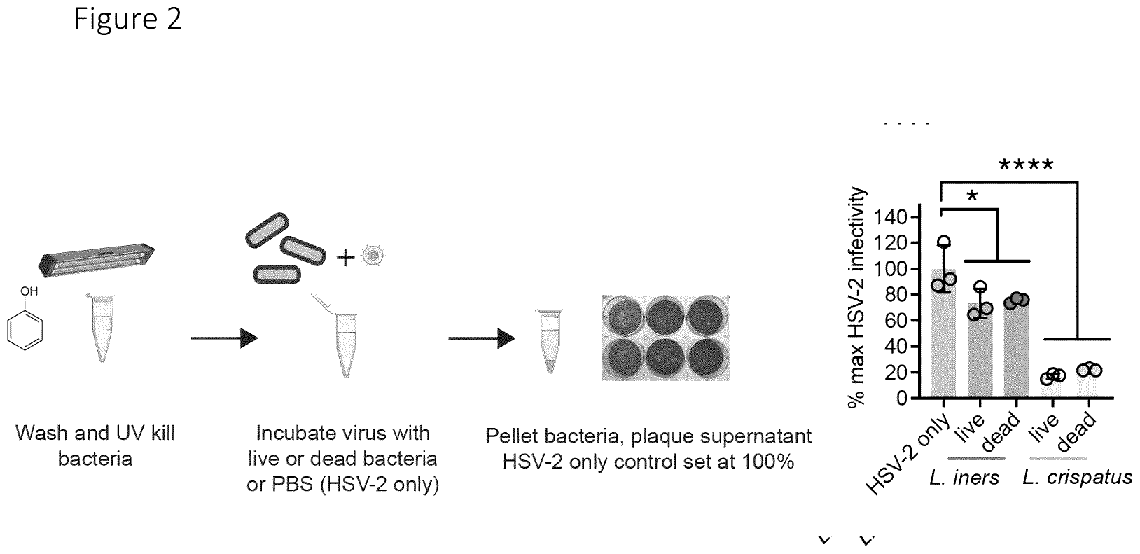

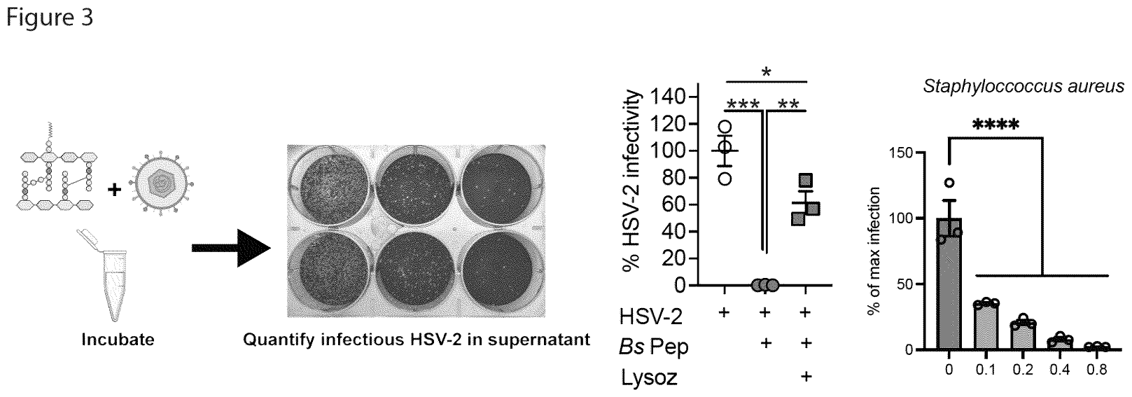

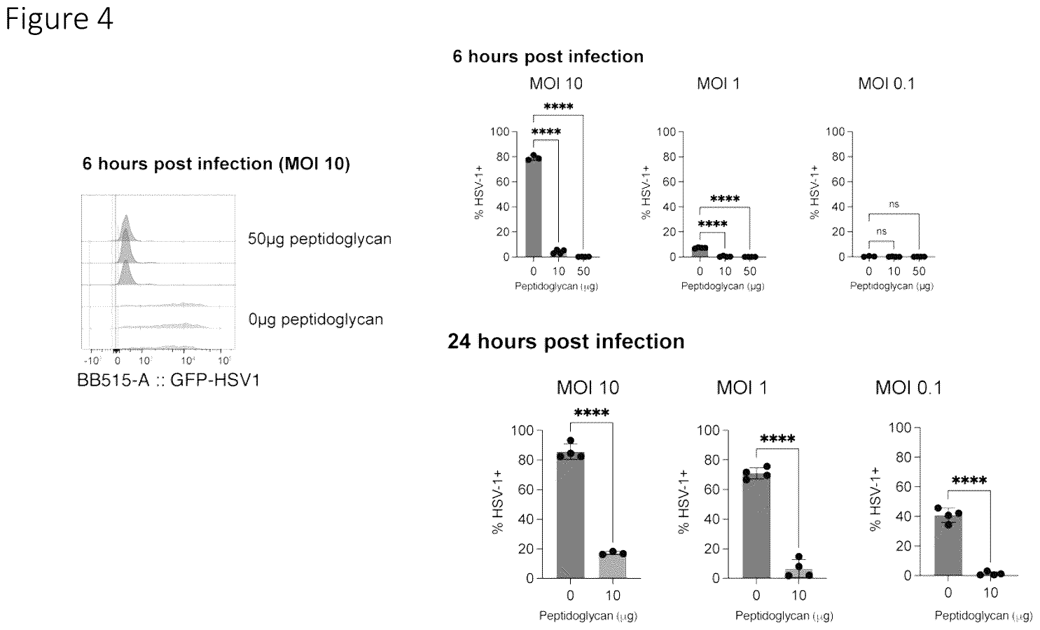

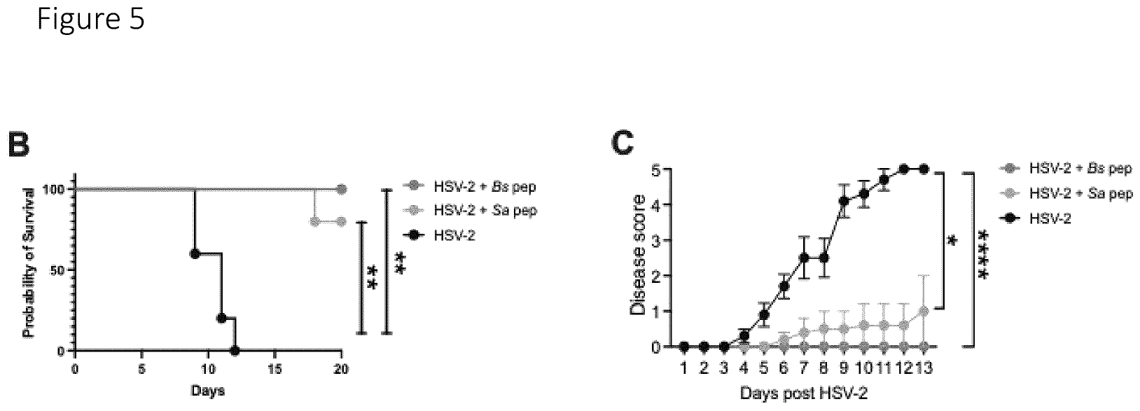

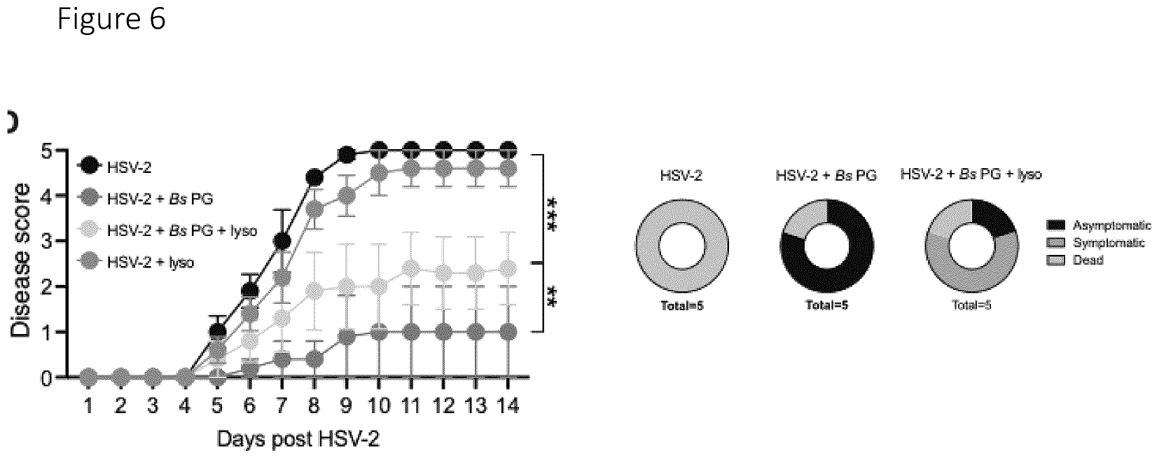

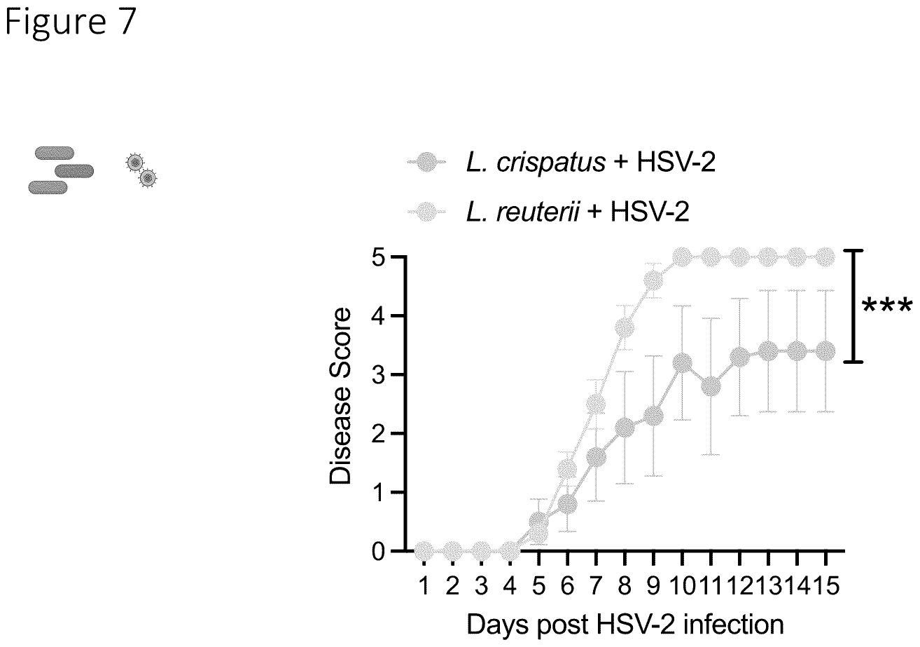

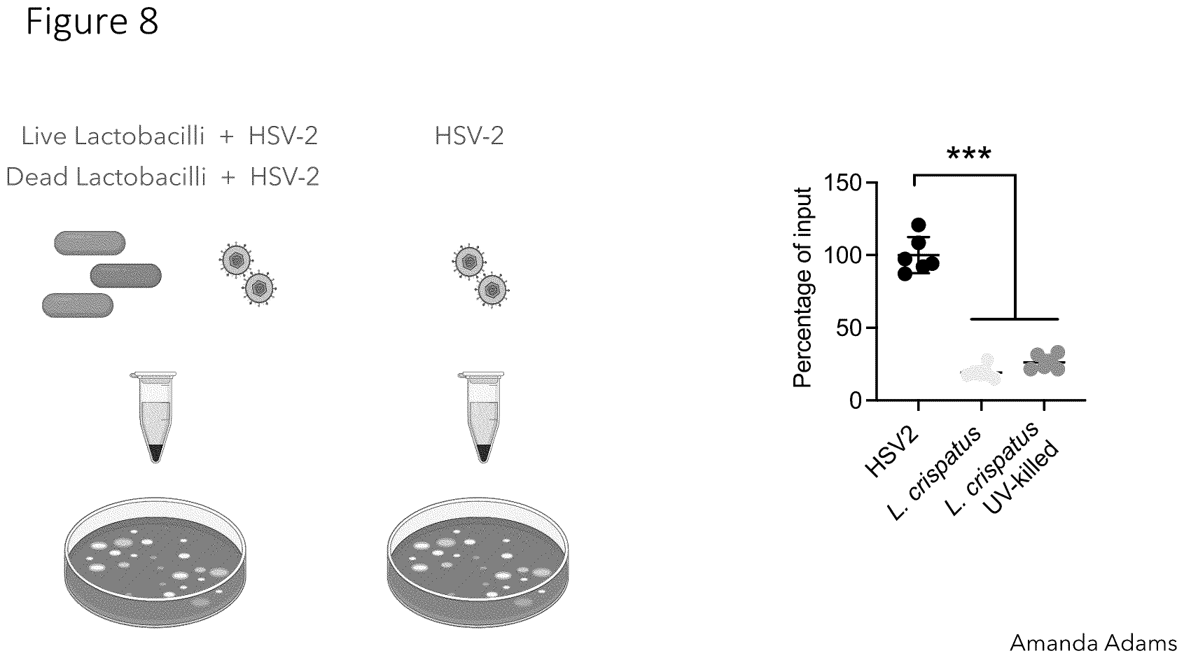

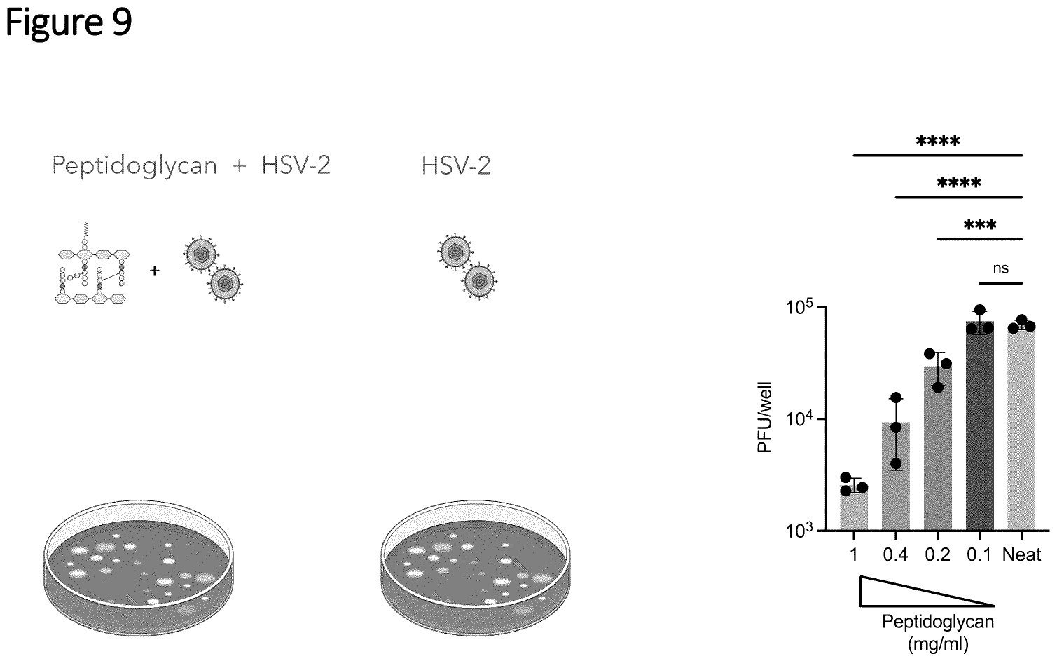

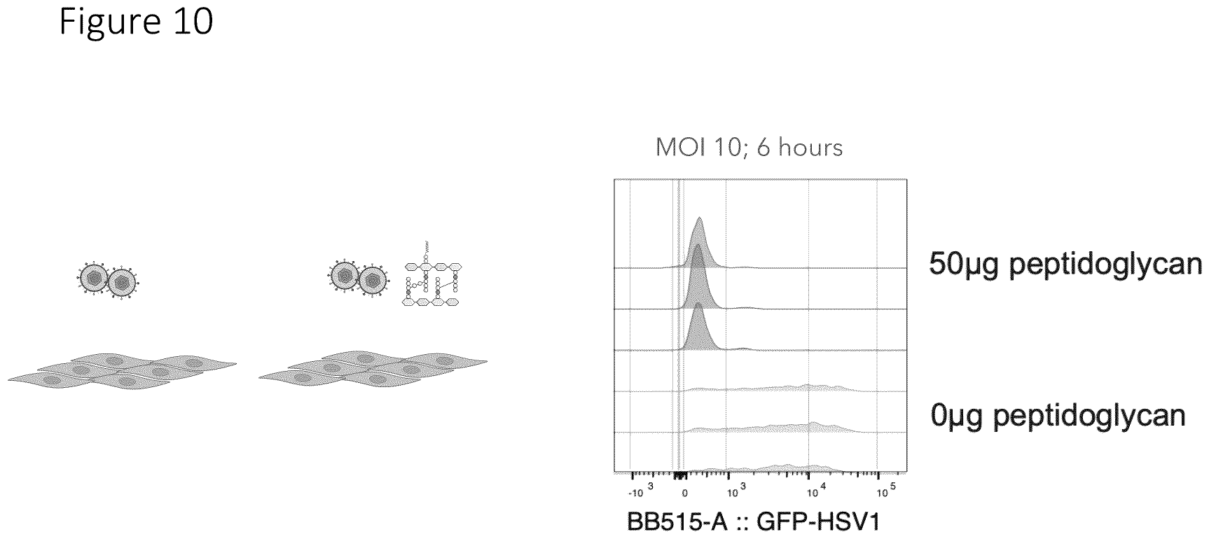

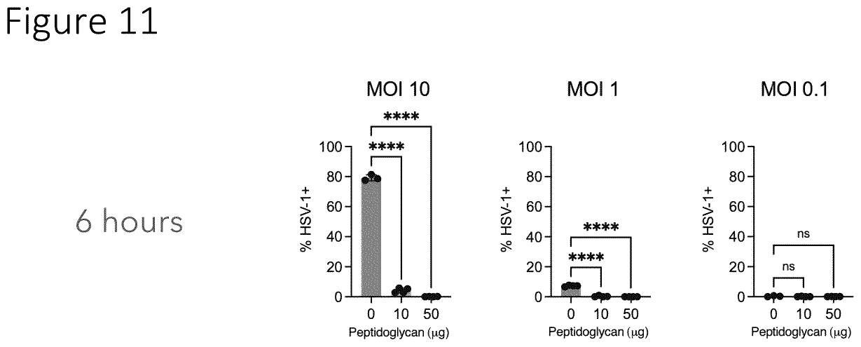

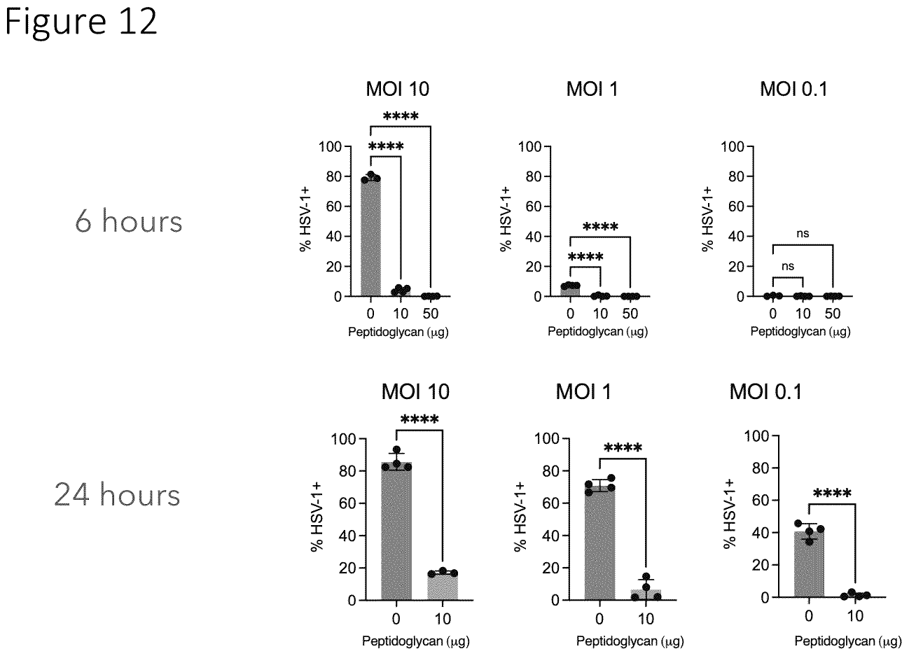

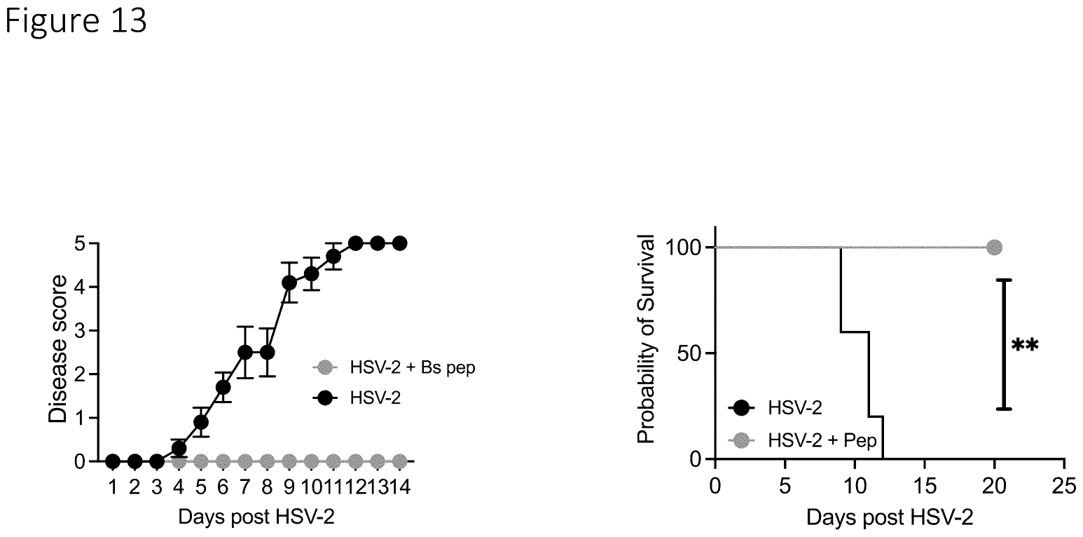

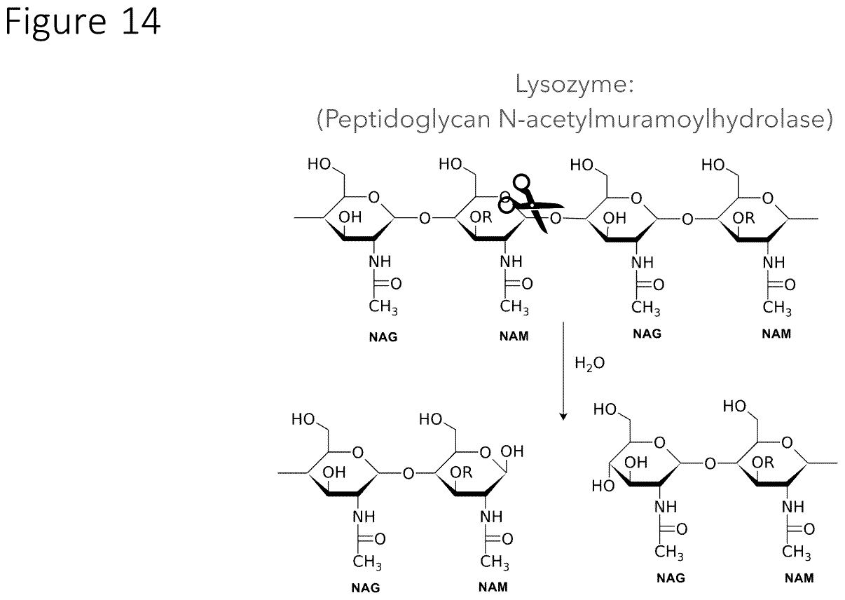

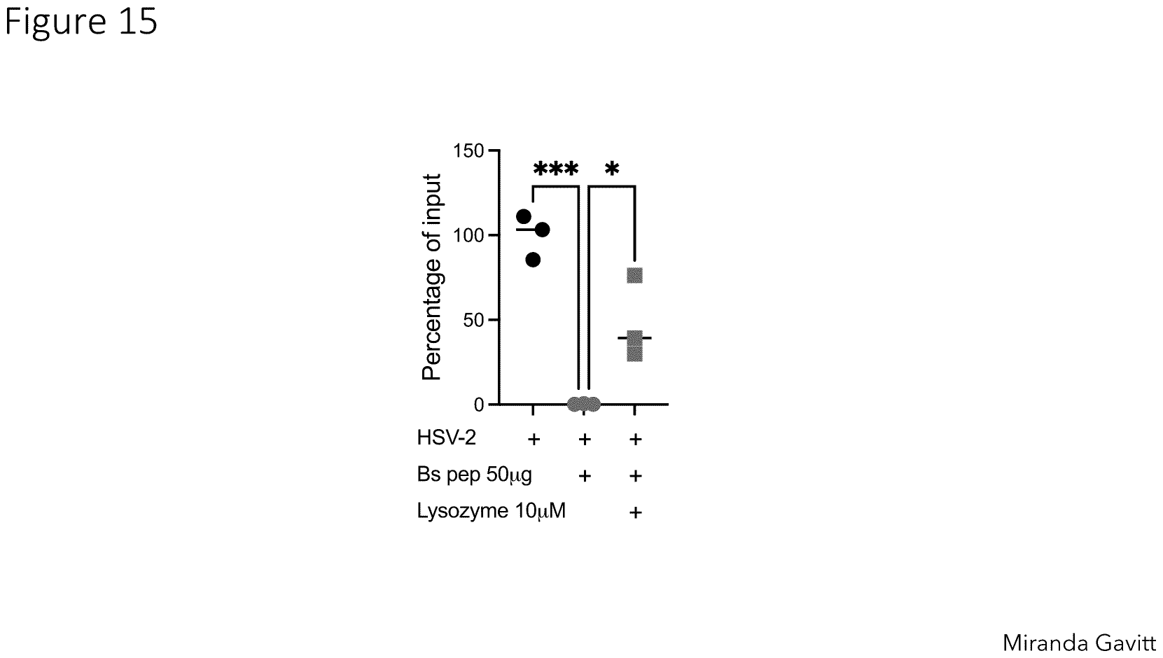

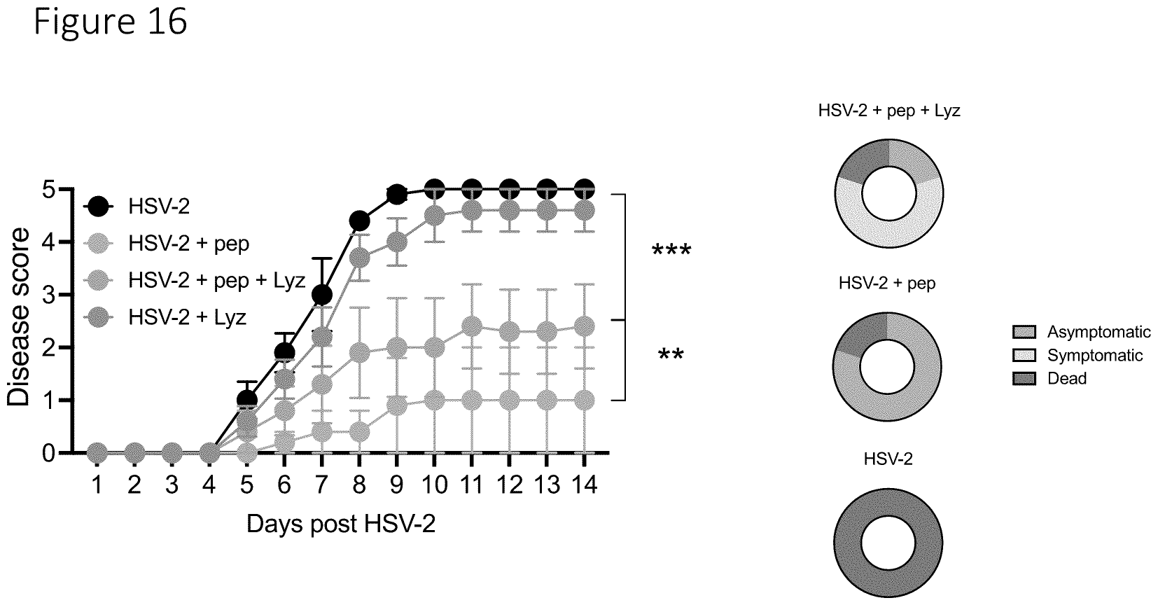

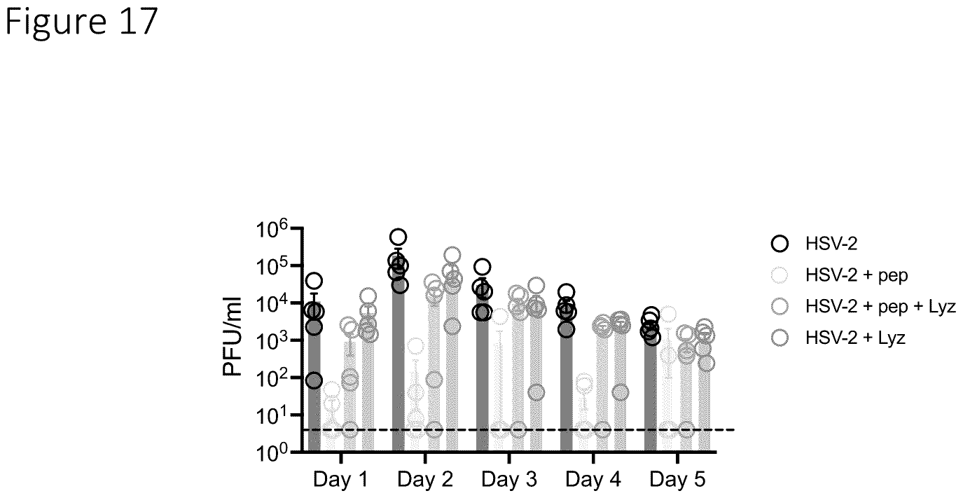

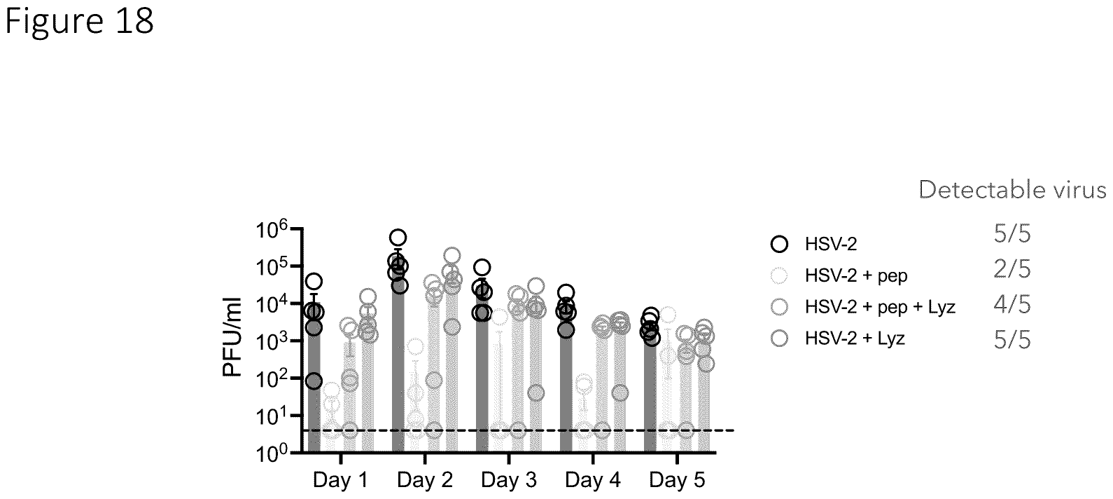

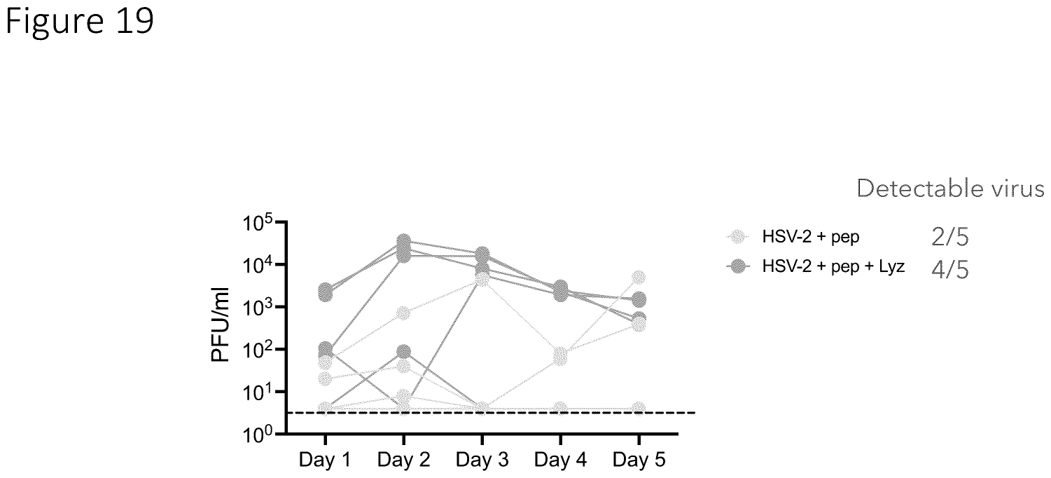

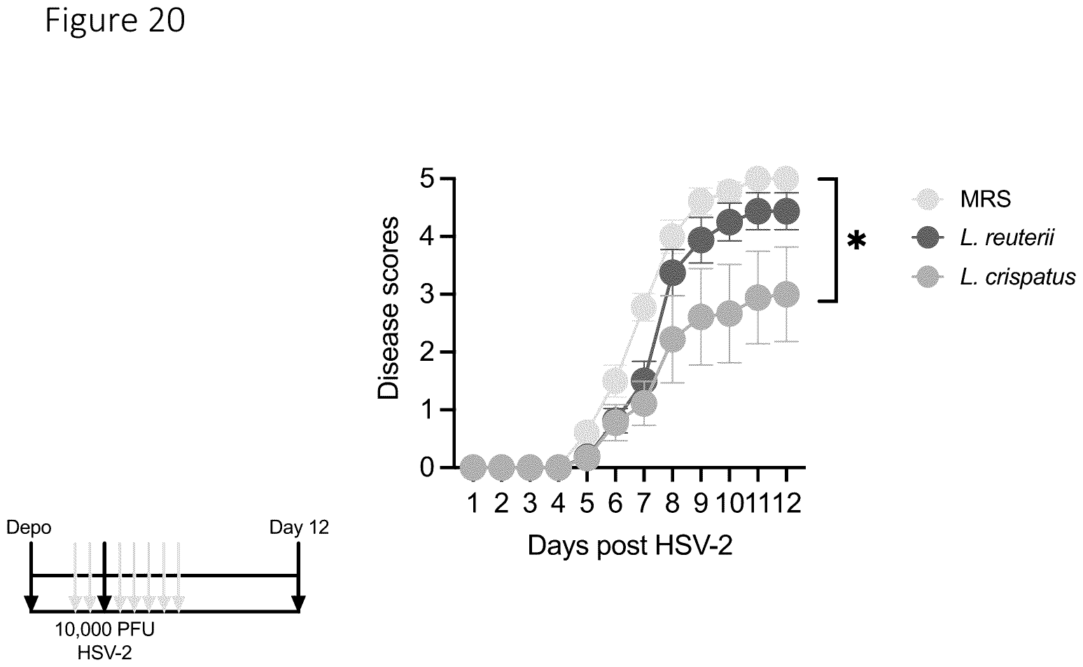

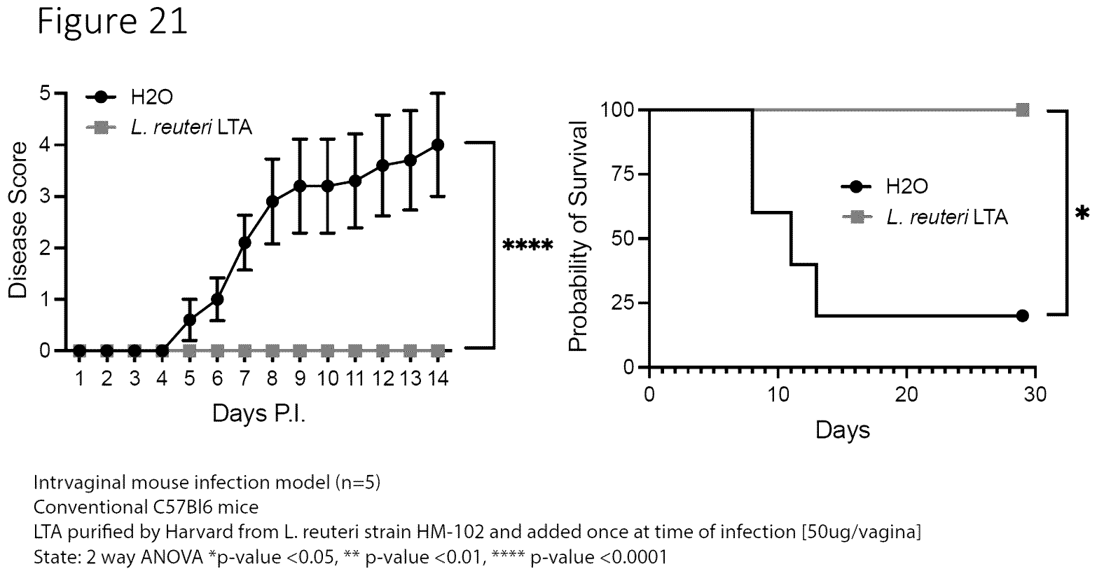

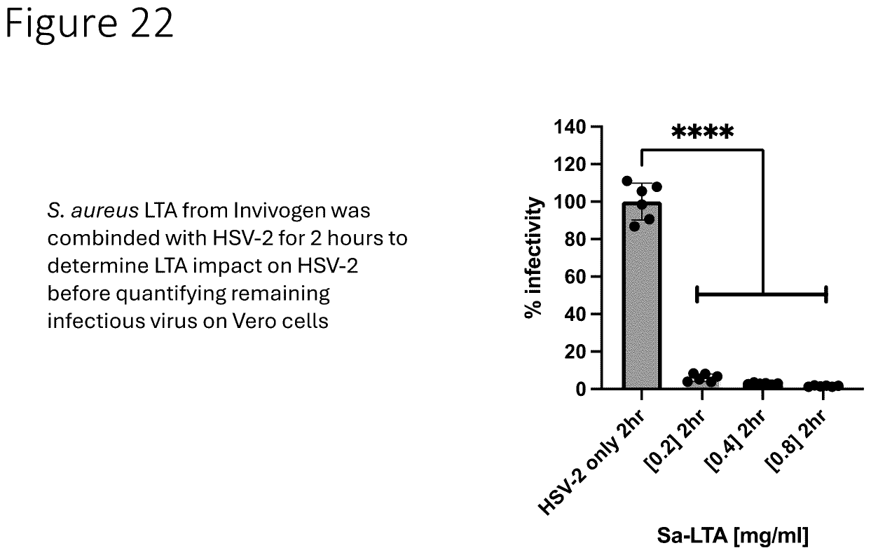

### DETAILED DESCRIPTION

The methods and compositions provided herein are based, in part, on the discovery that topical application of bacterial-derived peptidoglycans inhibits/reduces transmission of HSV viruses, including HSV-1 (commonly associated with cold sores and oral lesions) and HSV-2 (commonly associated with genital herpes). This could be especially important for preventing transmission of these viruses to newborns during birth. Longer chain peptidoglycans are more effective at reducing/preventing re-infection, as evidenced by the data disclosed herein, which show that peptidoglycans that have been treated with lysozyme lose the ability to prevent re-infection. In addition, peptidoglycans derived from vaginal lactobacilli are more effective than those derived from intestinal bacteria.

Herpes simplex virus 1 and 2, also known by their taxonomic names Human alphaherpesvirus 1 and Human alphaherpesvirus 2, are two members of the human Herpesviridae family, a set of viruses that produce viral infections in the majority of humans. Herpes simplex virus is composed of linear dsDNA, an icosahedral capsid that is 100 to 110 nm in diameter, with a spikey envelope.

In general, the pathogenesis of HSV-1 infection follows a cycle of primary infection of epithelial cells, latency primarily in neurons, and reactivation. HSV-1 is responsible for establishing primary and recurrent vesicular eruptions, primarily in the orolabial and genital mucosa. HSV-1 infection has a wide variety of presentations, including orolabial herpes, herpetic sycosis (HSV folliculitis), herpes gladiatorum, herpetic whitlow, ocular HSV infection, herpes encephalitis, Kaposi varicelliform eruption (eczema herpeticum), and severe or chronic HSV infection. Provided herein are methods of preventing or treating a HSV infection, such as orolabial herpes, herpetic sycosis (HSV folliculitis), herpes gladiatorum, herpetic whitlow, ocular HSV infection, herpes encephalitis, Kaposi varicelliform eruption (eczema herpeticum), and/or severe or chronic HSV infection in a subject.

HSV-1 infection can also occur on the digits or periungual, causing herpetic whitlow. Herpetic whitlow presents as deep blisters that may secondarily erode. Herpetic whitlow can also lead to lymphadenopathy of the epitrochlear or axillary lymph nodes in association with lymphatic streaking, mimicking bacterial cellulitis.

HSV-1 infection of the eye leads to ocular HSV in children and adults. Primary ocular HSV presents with keratoconjunctivitis that can be unilateral or bilateral. There can be associated eyelid tearing, edema, photophobia, chemosis (swelling of the conjunctiva), and preauricular lymphadenopathy. It is common for patients to experience recurrence, and in these cases, it is usually unilateral. Ocular HSV is a common cause of blindness in the United States when it manifests as keratitis or a branching dendritic corneal ulcer (which is pathognomonic for ocular HSV).

Provided herein are methods of treating or preventing an HSV infection, reducing the infectivity of such an infection, and/or reducing or preventing the transmission of a HSV infection or one of its pathological manifestations, such as orolabial herpes, herpetic sycosis (HSV folliculitis), herpes gladiatorum, herpetic whitlow, ocular HSV infection, herpes encephalitis, Kaposi varicelliform eruption (eczema herpeticum), and/or neonatal herpes viral infection by administering to a subject in need thereof a composition disclosed herein. The composition may comprise a replication competent or incompetent bacteria listed in Table 1 or 2, and/or the composition may comprise a peptidoglycan or LTA isolated or derived from a bacteria disclosed herein (a bacteria listed in Table 1 or 2).

Herpes encephalitis is a severe, typically fatal (mortality is greater than 70% if untreated) infection caused by HSV-1. It primarily affects the temporal lobe of the brain leading to focal neurological deficits localized to the temporal lobe.

Kaposi varicelliform eruption, or eczema herpeticum, presents as an extensive spreading of HSV infection in the setting of a compromised skin barrier (e.g., atopic dermatitis, Darier disease, pemphigus foliaceous, pemphigus vulgaris, Hailey-Hailey disease, mycosis fungoides, ichthyosis). Patients will display 2 to 3 mm punched-out erosions with hemorrhagic crusts in widespread distribution.

Neonatal herpes virus presents at day 5 to 14 of life and favors the scalp and the trunk. It may present with disseminated cutaneous lesions and involvement of oral and ocular mucosa. Central nervous system (CNS) involvement may occur and manifest as encephalitis with lethargy, poor feeding, bulging fontanelle, irritability, and seizures.

In the immunocompromised patient population, HSV infection can result in severe and chronic infection. The most common presentation of severe and chronic HSV infection is quickly enlarging ulcerations or verrucous/pustular lesions. It is not uncommon for patients to have respiratory or gastrointestinal tract involvement and present with dyspnea or dysphagia.

Herpes genitalis can be caused by the herpes simplex virus type 1 or 2, manifesting as a primary or recurrent infection. Most commonly, viral replication occurs in epithelial tissue and establishes dormancy in sensory neurons, reactivating periodically as localized recurrent lesions. It remains one of the most common sexually transmitted infections (STI).

### Definitions

For convenience, certain terms employed in the specification, examples, and appended claims are collected here.

The articles “a” and “an” are used herein to refer to one or to more than one (i.e. to at least one) of the grammatical object of the article. By way of example, “an element” means one element or more than one element.

The term “agent” is used herein to denote a chemical compound, a small molecule, a mixture of chemical compounds and/or a biological macromolecule (such as a nucleic acid, an antibody, an antibody fragment, a protein or a peptide). The activity of such agents may render them suitable as a “therapeutic agent” which is a biologically, physiologically, or pharmacologically active substance (or substances) that acts locally or systemically in a subject.

As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

As used herein, any two agents and/or additional agents may be conjointly administered according to the methods provided herein.

As used herein “derived” includes, but is not limited to, compounds that have been developed by synthetic methods (e.g., through native chemical ligation, Staudinger ligation, or other (orthogonal or otherwise) chemical reactions to couple synthetic peptides) but are substantially similar in structure to those found in bacteria. The term also includes compounds that have undergone methods of purifying or separating a compound from its original source, such that the compound is physically separated from its state found in nature, such as in or part of a cell or bacteria.

As used herein “isolated” is meant to encompass compounds that have undergone methods of purifying or separating a compound from its original source, such that the compound is separated from its state found in nature, such as in or part of a cell or bacteria.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The terms “prevent,” “preventing,” “prevention,” and the like refer to reducing the probability of developing a disease, disorder, or condition in a subject, who does not have, but is at risk of or susceptible to developing a disease, disorder, or condition.

As used herein, the term “subject” means a human or non-human animal selected for treatment or therapy. As used herein, “subject” may be a subject that is infected with HSV or a subject that is not infected with HSV. An infected subject may be exhibiting symptoms of active infection or may not show any symptoms of active infection. For example, the disclosure herein includes subjects that are uninfected, such as methods of preventing HSV transmission from an infected subject. An infected subject includes those with an active infection and those harboring dormant HSV. “Subject” includes newborn and infant humans.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment. For example, the peptidoglycan or bacterium disclosed herein may be administered in an amount to reduce HSV transmission or infectivity. Similarly, the peptidoglycan, LTA, or bacterium disclosed herein may be administered in an amount to reduce HSV symptoms.

“Treating” a disease in a subject or “treating” a subject having a disease refers to subjecting the subject to a pharmaceutical treatment, e.g., the administration of a drug, such that at least one symptom of the disease is decreased or prevented from worsening.

### Pharmaceutical Compositions

In certain embodiments, provided herein is a composition, e.g., a pharmaceutical composition, containing at least one bacterium described herein and/or at least one peptidoglycan derived from a bacterium described herein together with a pharmaceutically acceptable carrier. In one embodiment, the composition includes a combination of multiple (e.g., two or more) agents described herein. The bacterium may be a Lactobacillus bacteria, such as a bacteria from a strain listed in Table 1.

The composition may comprise a replication competent or incompetent bacteria disclosed herein (e.g., such as a species or strain listed in Table 1 or 2), and/or the composition may comprise a peptidoglycan or LTA isolated or derived from a bacteria disclosed herein (e.g., a bacteria species or strain listed in Table 1 or 2).

TABLE 1
Exemplary Lactobacillus
Lactobacillus crispatus Strain information

| Accession no. | ID |
|---|---|
| NKLQ00000000 | RL03 |
| NKLP00000000 | RL05 |
| NKLO00000000 | RL06 |
| NKLM00000000 | RL08 |
| NKLL00000000 | RL09 |
| NKLK00000000 | RL10 |
| NKLJ00000000 | RL11 |
| NKLF00000000 | RL16 |
| NKKX00000000 | RL26 |
| NKKW00000000 | RL27 |
| NKKU00000000 | RL29 |
| NKKR00000000 | RL32 |
| NKLR00000000 | RL02 |
| NKLN00000000 | RL07 |
| NKLI00000000 | RL13 |
| NKLH00000000 | RL14 |
| NKLG00000000 | RL15 |
| NKLE00000000 | RL17 |
| NKLD00000000 | RL19 |
| NKLC00000000 | RL20 |
| NKLB00000000 | RL21 |
| NKLA00000000 | RL23 |
| NKKZ00000000 | RL24 |
| NKKY00000000 | RL25 |
| NKKV00000000 | RL28 |
| NKKT00000000 | RL30 |
| NKKS00000000 | RL31 |
| NKKQ00000000 | RL33 |

| Additional | | |
|---|---|---|
| Exemplary Strains | Species | BEI HM-# |
| JV-V03 | L. gasseri | HM-104 |
| EX336960VC01 | L. gasseri | HM-398 |
| EX336960VC02 | L. gasseri | HM-399 |
| EX336960VC03 | L. gasseri | HM-400 |
| EX336960VC06 | L. gasseri | HM-403 |
| EX336960VC07 | L. gasseri | HM-404 |
| EX336960VC13 | L. gasseri | HM-407 |
| EX336960VC15 | L. gasseri | HM-409 |
| EX336960VC16 | L. gasseri | HM-410 |
| MV-22 | L. gasseri | HM-644 |
| 224-1 | L. gasseri | HM-647 |
| PSS7772D | L. gasseri | HM-1278 |
| JV-V16 | L. jensenii | HM-105 |
| EX849587VC03 | L. jensenii | HM-372 |
| EX849587VC06 | L. jensenii | HM-374 |
| 115-3-CHN | L. jensenii | HM-640 |
| 269-3 | L. jensenii | HM-645 |
| 208-1 | L. jensenii | HM-646 |
| EX849587VC02 | L. crispatus | HM-371 |
| EX849587VC04 | L. crispatus | HM-373 |
| EX849587VC07 | L. crispatus | HM-375 |
| EX849587VC08 | L. crispatus | HM-376 |
| | L. crispatus | HM-637 |
| | L. crispatus | HM-636 |
| | L. crispatus | HM-638 |
| | L. crispatus | HM-103 |
| | L. reuteri | HM-102 |
| | L. rhamnosus | HM-106 |
| Lactin V 03(V1)-b | L. iners | HM-701 |
| | L. iners | HM-702 |
| SPIN 2503V10-D | L. iners | HM-704 |
| | L. iners | HM-705 |
| | L. iners | HM-706 |
| | L. iners | HM-707 |
| | L. iners | HM-708 |
| | L. iners | HM-126 |
| | L. iners | HM-131 |
| | ASF 360 | |
| | ASF 361 | |

TABLE 2
Exemplary S. aureus
Staphylococcus aureus Strain information

ID
NCTC 8325
Newman
ATCC 25923
ATCC 6538
RN4220
Mu50
ATCC 29213
JKD6008
MW2 (USA400)
SH1000
USA100
USA200
USA300

In some aspects, provided herein are methods of treating or preventing the transmission of Herpes Simplex Viruses (HSV) in a subject, the method comprising administering to the subject a composition comprising a peptidoglycan (e.g., an isolated or purified peptidoglycan). The peptidoglycan may be derived or isolated from a bacteria. The bacteria may be a lactobacilli, such as a vaginal lactobacilli or intestinal lactobacilli. In some embodiments, the lactobacilli are Lactobacillus crispatus. In some embodiments, the lactobacilli are Lactobacillus reuteri. In some embodiments, the lactobacilli are any species or strain listed in Table 1. In some embodiments, the bacteria are any species or strain listed in Table 2.

The basic structure of peptidoglycan contains a carbohydrate backbone of alternating units of N-acetylglucosamine (GlcNAc) and N-acetylmuramic acid, with the N-acetylmuramic acid residues cross-linked to peptides. In some embodiments, a peptidoglycan is at least 10 disaccharide units long. In some embodiments, the peptidoglycan is at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 disaccharide units long.

In some embodiments, the peptidoglycan is no more than 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 disaccharide units long.

In some embodiments, the peptidoglycan is between 10 and 1000 disaccharide units long. In some embodiments, the peptidoglycan is between 20 and 900 disaccharide units long. In some embodiments, the peptidoglycan is between 50 and 800 disaccharide units long. In some embodiments, the peptidoglycan is between 75 and 500 disaccharide units long. In some embodiments, the peptidoglycan is between 100 and 400 disaccharide units long. In some embodiments, the peptidoglycan is between 100 and 300 disaccharide units long. In some embodiments, the peptidoglycan is between 150 and 250 disaccharide units long. In some embodiments, the peptidoglycan is between 200 and 500 disaccharide units long. In some embodiments, the peptidoglycan is between 200 and 300 disaccharide units long. In some embodiments, the peptidoglycan is between 250 and 350 disaccharide units long. In some embodiments, the peptidoglycan is between 100 and 150 disaccharide units long. In some embodiments, the peptidoglycan is between 150 and 200 disaccharide units long.

In some aspects, provided herein are methods of treating or preventing the transmission of Herpes Simplex Viruses (HSV) in a subject, the method comprising administering to the subject a composition comprising a lipoteichoic acid (e.g., an isolated or purified lipoteichoic acid). The peptidoglycan may be derived or isolated from a bacteria. The bacteria may be a lactobacilli, such as a vaginal lactobacilli or intestinal lactobacilli. In some embodiments, the bacteria are S. aureus. In some embodiments, the lactobacilli are Lactobacillus reuteri. In some embodiments, the lactobacilli are any species or strain disclosed herein (e.g., any strain listed in Table 1 or Table 2).

In some embodiments, the pharmaceutical composition is delivered locally or systemically.

In some embodiments, the agent or pharmaceutical composition is administered with an additional anti-viral therapeutic agent, e.g. an anti-viral that targets HSV. In certain embodiments, the additional therapeutic agent is acyclovir, valacyclovir, or famciclovir.

The pharmaceutical compositions and/or agents disclosed herein may be specially formulated for administration in solid or liquid form, including those adapted for the following: (1) topical administration; (2) oral administration, for example, drenches (aqueous or non-aqueous solutions or suspensions), tablets, e.g., those targeted for buccal, sublingual, and systemic absorption, boluses, powders, granules, pastes for application to the tongue; or (3) parenteral administration, for example, by subcutaneous, intramuscular, intravenous, intrathecal, intracerebral or epidural injection as, for example, a sterile solution or suspension, or sustained-release formulation. Methods of preparing pharmaceutical formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

In some embodiments, the topical formulation is an emulsion, cream, lotion, gel, oil, ointment, aerosol spray, or semi-solid formulation. In some embodiments, the topical formulation comprises a carrier, wherein said carrier is selected from the group consisting of trehalose, malto-dextrin, rice flour, micro-crystalline cellulose, magnesium stearate, inositol, fructo-oligosaccharide, gluco-oligosaccharide, dextrose, sucrose, talc, water, physiological salt solution, urea, methanol, ethanol, propanol, butanol, ethylene glycol, propylene glycol, white pertrolatum, isopropyl myristate, lanolin, lanolin alcohol, mineral oil, lavender oil, nasturtium extract oil, sorbitan mono-oleate, cetylstearyl alcohol, hydroxypropyl cellulose, detergent, sucrose stearate, sucrose cocoate, sucrose distearate, 2-ethyl-1,3-hexanediol, polyoxypropylene-15-stearyl ether, glycerol stearate, glycerin, synthetic spermaceti, cetyl alcohol, butylparaben, propylparaben, and methylparaben.

In some embodiments, the invention relates to a method for treating a skin condition in a subject in need thereof, comprising applying to the skin of the subject a topical formulation. In some embodiments, the invention relates to a method for treating a mucous membrane condition in a subject in need thereof, comprising applying to a mucous membrane of the subject a topical formulation.

Exemplary identities of various constituents of the topical formulations of some embodiments of the present invention are described below.

### Vehicles

Suitable topical vehicles and vehicle components for use with the formulations of the invention are well known in the cosmetic and pharmaceutical arts, and include such vehicles (or vehicle components) as water; organic solvents such as alcohols, glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, vuvla, or vagina, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In some embodiments, formulations do not have methanol, ethanol, propanols, or butanol.

### Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄₀)ₐ(CH(CH₃)CH₂₀)_{b}(C₂H₄O)ₐH, wherein a is about 101 and b is about 56)). Appropriate combinations or mixtures of such surfactants may also be used according to the present invention. Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### Moisturizers, Emollients, and Humectants

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800. Suitable emollients or humectants for use in the formulations of the present invention include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl) stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, and 1,3-bis(N-2-(hydroxyethyl) palmitoylamino)-2-hydroxypropane. In addition, appropriate combinations and mixtures of any of these moisturizing agents and emollients may be used in accordance with the present invention.

### Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols, tocopheryl acetate, sodium ascorbate/ascorbic acid, ascorbyl palmitate, propyl gallate, and chelating agents like EDTA (e.g., disodium EDTA), citric acid, and sodium citrate.

In some embodiments, the antioxidant or preservative comprises (3-(4-chlorophenoxy)-2-hydroxypropyl) carbamate.

In some embodiments, antioxidants or preservatives of the present invention may also function as a moisturizer or emollient, for example.

In addition, combinations or mixtures of these preservatives or anti-oxidants may also be used in the formulations of the present invention.

### Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

### Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, anti-irritants, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate). For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Also provided herein are methods that further comprise administering an antibacterial or antimicrobial agent prior to or conjointly with a bacteria or composition disclosed herein.

### Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

### Kits

Also provided herein are kits comprising (a) a composition comprising a peptidoglycan disclosed herein and (b) an additional agent for the prevention of HSV selected from acyclovir, valacyclovir, or famciclovir. In some embodiments, the peptidoglycan is isolated or derived from L. crispatus bacterium. The HSV may be HSV-1 or HSV-2.

In some embodiments, a peptidoglycan is at least 10 disaccharide units long. In some embodiments, the peptidoglycan is at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 disaccharide units long.

In some embodiments, the peptidoglycan is no more than 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 disaccharide units long.

In some embodiments, the peptidoglycan is between 10 and 1000 disaccharide units long. In some embodiments, the peptidoglycan is between 20 and 900 disaccharide units long. In some embodiments, the peptidoglycan is between 50 and 800 disaccharide units long. In some embodiments, the peptidoglycan is between 75 and 500 disaccharide units long. In some embodiments, the peptidoglycan is between 100 and 400 disaccharide units long. In some embodiments, the peptidoglycan is between 100 and 300 disaccharide units long. In some embodiments, the peptidoglycan is between 150 and 250 disaccharide units long. In some embodiments, the peptidoglycan is between 200 and 500 disaccharide units long. In some embodiments, the peptidoglycan is between 200 and 300 disaccharide units long. In some embodiments, the peptidoglycan is between 250 and 350 disaccharide units long. In some embodiments, the peptidoglycan is between 100 and 150 disaccharide units long. In some embodiments, the peptidoglycan is between 150 and 200 disaccharide units long.

### Therapeutic Methods

In some aspects, provided herein are methods of treating or preventing the transmission of HSV by administering to a subject a pharmaceutical composition described herein. The transmission may be from mother to newborn, for example during a vaginal delivery (e.g., applied topically to the vagina).

Also provided herein are methods of treating or preventing the transmission of or reducing the infectivity of Herpes Simplex Virus (HSV) in a subject, the method comprising administering to the subject a composition comprising a peptidoglycan (e.g., an isolated or purified peptidoglycan).

In some embodiments, the peptidoglycan reduces the infectivity by at least 1% 2% 3% 4% 5% 6% 7% 8% 9% 10% 11% 12% 13% 14% 15% 16% 17% 18% 19% 20% 21% 22% 23% 24% 25% 26% 27% 28% 29% 30% 31% 32% 33% 34% 35% 36% 37% 38% 39% 40% 41% 42% 43% 44% 45% 46% 47% 48% 49% 50% 51% 52% 53% 54% 55% 56% 57% 58% 59% 60% 61% 62% 63% 64% 65% 66% 67% 68% 69% 70% 71% 72% 73% 74% 75% 76% 77% 78% 79% 80% 81% 82% 83% 84% 85% 86% 87% 88% 89% 90% 91% 92% 93% 94% 95% 96% 97% 98% 99%, or 100%. As used herein, “reducing the infectivity” of a virus can apply to the percent reduction of transmission between an individual receiving a composition disclosed herein and a person not receiving a composition, or receiving a negative control. It can also encompass the reduction in infectivity statistically between a treatment population and a non-treated or control population.

In other aspects, provided herein are methods of preventing or treating a Herpes Simplex Viruses (HSV) infection in a subject in need thereof, the method comprising administering to the subject a composition comprising a peptidoglycan (e.g., an isolated or purified peptidoglycan).

Also provided herein are methods of treating or preventing the transmission of or reducing the infectivity of Herpes Simplex Virus (HSV) in a subject, the method comprising administering to the subject composition comprising a Lactobacillus bacterium (such as a bacterium from a Lactobacillus crispatus strain listed in Table 1).

Also provided herein are methods of treating or preventing the transmission of or reducing the infectivity of Herpes Simplex Virus (HSV) in a subject, the method comprising administering to the subject a composition comprising a bacterium species or strain listed in Table 1.

The bacterium may be replication competent (capable of replicating). The bacterium may not be replication competent (e.g., it may be killed bacteria). In some embodiments, the bacterium are lactobacilli are any species or strain listed in Table 1 or Table 2.

Also provided herein are methods of treating or preventing the transmission of or reducing the infectivity of Herpes Simplex Virus (HSV) in a subject, the method comprising administering to the subject a composition comprising a lipoteichoic acid (e.g., a lipoteichoic acid is isolated or derived from a L. reuteri or S. aureus bacterium.

In some embodiments, the HSV is HSV-1 or HSV-2. In some embodiments, the composition is formulated for topical delivery. The composition may comprise a pharmaceutically acceptable carrier. In some embodiments, the method further comprises administering (e.g., conjointly administering) an additional agent for the treatment or prevention of HSV. In some embodiments, the method may comprise administering (e.g., conjointly administering) a Lactobacillus bacterium (e.g., any Lactobacillus bacterium disclosed herein) and a peptidoglycan to a subject.

As disclosed herein, in any embodiment, the Lactobacillus bacterium may be a L. crispatu, L. gasseri, L. jensenii, L. reuteri, L. rhamnosus, or an L. iners bacterium.

In some embodiments, the subject has HSV-1 and/or HSV-2.

Actual dosage levels of the active ingredients in the pharmaceutical compositions or agents to be administered may be varied so as to obtain an amount of the active ingredient (e.g., an agent described herein) which is effective to achieve the desired therapeutic response for a particular patient, composition, and mode of administration, without being toxic to the patient.

The compositions disclosed herein may be administered over any period of time effective to achieve the desired therapeutic response for a particular patient, composition, and mode of administration, without being toxic to the patient. For example, the dose may be administered regularly once a patient becomes pregnant to reduce the transmission of HSV to the newborn. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day. In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months, for at least 1 year, for at least two years, at least three years, or at least five years.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could prescribe and/or administer doses of the compounds employed in the pharmaceutical composition at levels lower than that required in order to achieve the desired therapeutic effect and gradually increase the dosage until the desired effect is achieved.

### Exemplification

Exemplary methods are provided in separate documents filed herewith.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing the transmission of Herpes Simplex Virus (HSV) in a subject, the method comprising administering to the subject a composition comprising a peptidoglycan.

2. The method of claim 1, wherein the peptidoglycan is isolated or derived from a lactobacilli bacteria.

3. The method of claim 2, wherein the lactobacilli bacteria is a vaginal lactobacilli or intestinal lactobacilli.

4. The method of claim 1, wherein the peptidoglycan is isolated or derived from L. crispatus bacterium.

5. The method of claim 1, wherein the peptidoglycan is at least 10 disaccharide units long.

6. (canceled)

7. The method of claim 1, wherein the composition is formulated for topical delivery.

8. (canceled)

9. A method of treating or preventing the transmission of Herpes Simplex Virus (HSV) in a subject, the method comprising administering to the subject a composition comprising a L. crispatus bacterium.

10. The method of claim 9, wherein the HSV is HSV-1 or HSV-2.

11. The method of claim 9, wherein the composition is formulated for topical delivery.

12. The method of claim 1, wherein the composition comprises a pharmaceutically acceptable carrier.

13. The method of claim 1, wherein the method further comprises administering an additional agent for the prevention of HSV.

14. The method of claim 9, the method comprising administering to the subject a composition comprising a lipoteichoic acid.

15. The method of claim 14, wherein the lipoteichoic acid is isolated or derived from a L. reuteri bacterium S. aureus bacterium.

16-17. (canceled)

18. The method of claim 14, wherein the composition is formulated for topical delivery.

19. A kit comprising (a) a composition comprising a peptidoglycan and/or lipoteichoic acid and (b) an additional agent for the prevention of HSV selected from acyclovir, valacyclovir, or famciclovir.

20. The kit of any of claim 19, wherein the peptidoglycan is isolated or derived from L. crispatus bacterium.

21. The kit of claim 19, wherein the peptidoglycan is at least 10 disaccharide units long.

22. The kit of claim 19, wherein the HSV is HSV-1 or HSV-2.

23. (canceled)

24. The kit of any of claim 20, wherein the lipoteichoic acid is isolated or derived from L. reuteri bacterium or S. aureus bacterium.

25-26. (canceled)
